(19) United States
(12) Patent Application Publication
(10) Pub. No.: US 2026/0108460 A1
(43) Pub. Date: Apr. 23, 2026

(54) STABLE ORAL LIQUID COMPOSITIONS

(71) Applicant: Method Pharmaceuticals, LLC, Southlake, TX (US)

(72) Inventors: Jitesh Devendra, Chennai (IN); Sampathkumar Devarajan, Chennai (IN); Mathivanan Rajagopal, Chennai (IN); Bindumadavan Kannan, Chennai (IN); Ramesh Subramani, Chennai (IN); Jeevanantham Raji, Chennai (IN); Matthew Scott Tucker, Southlake, TX (US); Christopher Boone, Southlake, TX (US)

(21) Appl. No.: 19/365,720

(22) Filed: Oct. 22, 2025

Related U.S. Application Data
(60) Provisional application No. 63/711,144, filed on Oct. 23, 2024.

(51) Int. Cl. A61K 9/10; A61K 9/00; A61K 9/08; A61K 9/50; A61K 31/4422; A61K 31/55; A61K 47/10; A61K 47/12; A61K 47/18; A61K 47/26; A61K 47/32; A61K 47/38; A61K 47/40; A61K 47/46

(52) U.S. Cl. CPC A61K 9/10; A61K 9/0095; A61K 9/08; A61K 9/5042; A61K 31/4422; A61K 31/55; A61K 47/10; A61K 47/12; A61K 47/183; A61K 47/26; A61K 47/32; A61K 47/38; A61K 47/40; A61K 47/46

(57) ABSTRACT

The present disclosure relates to methods and pharmaceutical compositions of angiotensin converting enzyme inhibitor and calcium channel blocker, particularly stable oral liquid compositions including angiotensin converting enzyme inhibitor, calcium channel blocker and one or more pharmaceutically acceptable excipients for the treatment of hypertension, coronary artery disease, heart failure, acute myocardial infarction and other related cardiovascular diseases.

### CROSS-REFERENCE TO RELATED APPLICATIONS

This application claims the benefit of, and priority to, U.S. Provisional Patent Application No. 63/711,144, filed on Oct. 23, 2024, the entire contents of which are hereby incorporated herein by reference.

### FIELD OF THE INVENTION

The present invention provides a stable oral liquid formulation comprising angiotensin converting enzyme inhibitor, calcium channel blocker and one or more pharmaceutically acceptable excipients for the treatment of hypertension, coronary artery disease, heart failure, acute myocardial infarction and other related cardiovascular diseases.

### BACKGROUND OF THE INVENTION

Hypertension, or high blood pressure, is a serious health issue in many countries. Left unchecked, hypertension is considered a substantial risk factor for cardiovascular and other related diseases including coronary heart disease, myocardial infarction, congestive heart failure, stroke and kidney failure. Hypertension is classified as primary hypertension or secondary hypertension. Primary hypertension has no known cause and may be related to a number of environmental, lifestyle and genetic factors such as stress, obesity, smoking, inactivity and sodium intake. Secondary hypertension can be caused by drug or surgical interventions or by abnormalities in the renal, cardiovascular or endocrine system.

Many blood pressure medications, known as antihypertensives, are available by prescription to lower high blood pressure or hypertension. Various therapeutic classes of drugs are available and include alpha-adrenergic blockers, beta-adrenergic blockers, calcium channel blockers, hypotensives, mineralocorticoid antagonists, central alpha-agonists, diuretics and rennin-angiotensin-aldosterone inhibitors which include angiotensin II receptor antagonists and angiotensin converting enzyme inhibitors. Angiotensin converting enzyme inhibitors inhibit angiotensin converting enzyme, a peptidyl-dipeptidase that catalyzes angiotensin I to angiotensin II, a potent vasoconstrictor involved in regulating blood pressure.

It is currently known that angiotensin converting enzyme is an enzyme with multiple actions, that is, it acts in several substrates. Apart from acting as a dipeptidase in angiotensin I and in bradykinin, it is also capable of cleaving other peptides, indicating that the enzyme may act in several tissues and systems. An important advantage of angiotensin converting enzyme inhibitors is to prevent the harmful effects of angiotensin II in cardiac and vascular remodeling, being the antihypertensive class of highest efficacy in reducing left ventricular hypertrophy and vascular stiffness, also improving endothelial dysfunction. They are useful to heart failure patients with or without associated hypertension, also improving heart failure survival. So, they reduce cardiac remodeling in systolic heart failure and in post-acute myocardial infarction.

Angiotensin converting enzyme inhibitors are generally very difficult to formulate into dosage forms as certain angiotensin converting enzyme inhibitors upon contact with commonly used pharmaceutical excipients are prone to various types of degradation such as cyclization via internal nucleophilic attack to form substituted diketopiperazines, hydrolysis of the side-chain ester group and oxidation to form products having often unwanted coloration. New safe pharmaceutical compositions for the treatment of hypertension, coronary artery disease, heart failure, acute myocardial infarction and other related cardiovascular diseases are constantly being researched.

### SUMMARY

The oral liquid dosage form addresses patients that have difficulty in swallowing tablets. Liquids are easier to swallow than solid dosage forms and provides more rapid absorption leading to faster therapeutic response. Oral liquids offer dosing flexibility and can be easily administered to geriatric, neonatal and pediatric patients. While investigating oral solution/suspension dosage forms, it was discovered that stability depends on the inactive ingredients present in the formulation. The lack of dose uniformity may also result in an incorrect dosage amount, and thus, may be problematic for the treatment of a patient with hypertension and/or coronary artery disease at least because a satisfactory therapeutic effect may not be achieved.

Oral liquids may assist with better bioavailability because the drug is already distributed in solution/suspension dosage form, which provides homogenous system with less chance of dose variation. The liquid formulations of the current invention disclosed herein serve to solve the above-mentioned drawbacks and there are no formulations comprising combination of angiotensin converting enzyme inhibitor and calcium channel blocker available in the form of oral liquid in the market. Accordingly, there is a need for developing oral liquid pharmaceutical products comprising of angiotensin converting enzyme inhibitor, calcium channel blocker and other pharmaceutical excipients.

Therefore, described herein are the oral liquid pharmaceutical products comprising of angiotensin converting enzyme inhibitor, calcium channel blocker and other pharmaceutical excipients.

In embodiments, a composition for a stable oral liquid formulation is provided comprising benazepril or a pharmaceutically acceptable salt or solvate thereof; amlodipine or a pharmaceutically acceptable salt or solvate thereof; a sweetening agent; and a complexing agent.

In embodiments, the benazepril or a pharmaceutically acceptable salt or solvate thereof is in an amount of from about 0.3% to about 7% w/w; the amlodipine or a pharmaceutically acceptable salt or solvate thereof is in amount of from about 0.05% to about 2% w/w; the sweetening agent is in an amount of from about 0.5% to about 75% w/w; and the complexing agent is in an amount of from about 0.5% to about 50% w/w.

In further embodiments, a composition for a stable oral liquid formulation is provided including benazepril or a pharmaceutically acceptable salt or solvate thereof; amlodipine or a pharmaceutically acceptable salt or solvate thereof; a sweetening agent; a complexing agent; a viscosity modifier; and an anticaking agent.

In still further embodiments, a method of preparing a stable oral liquid pharmaceutical formulation is provided, including dissolving and or dispersing a complexing agent in a pharmaceutically acceptable solvent to obtain a first clear solution and/or a first uniform dispersion; adding a calcium channel blocker or a pharmaceutically acceptable salt thereof to the first clear solution and/or the first uniform dispersion and mixing until dissolution and/or uniform dispersion is achieved; adding a preservative and/or antioxidant to the first clear solution and/or the first uniform dispersion obtained and mixing until uniform solution or dispersion is achieved; dissolving or dispersing an angiotensin converting enzyme inhibitor or a pharmaceutically acceptable salt thereof in a solvent to obtain a second clear solution or a second uniform dispersion; separately dissolving or dispersing a viscosity modifier in a co-solvent to obtain a thickening solution or dispersion; combining the first and second clear solutions or the first and second uniform dispersion with the thickening solution or dispersion under continuous stirring to obtain a homogenous mixture; incorporating a sweetening agent to the homogenous mixture; and adjusting the final volume with a solvent to obtain a stable oral liquid formulation.

The compositions and their methods of manufacture disclosed herein relate to pharmaceutically acceptable stable liquid formulations comprising angiotensin converting enzyme inhibitor, calcium channel blocker and one or more pharmaceutically acceptable excipients, for oral administration, for the treatment of hypertension, coronary artery disease, heart failure, acute myocardial infarction and other related cardiovascular diseases.

The present invention relates to manufacturable and simultaneously storage-stable and bioavailable oral liquid formulations of angiotensin converting enzyme inhibitor, calcium channel blocker with one or more pharmaceutically acceptable excipients for cardiovascular and other related diseases. Further, stable oral liquid compositions of angiotensin converting enzyme inhibitor and calcium channel blocker provide methods of treating hypertension, heart failure, acute myocardial infarction, comprising administering to a patient in need thereof.

### DETAILED DESCRIPTION OF THE INVENTION

The following paragraphs detail various embodiments of the invention. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and combined with other important aspects of the invention described elsewhere within this specification as a whole and including the examples and figures. The skilled person will appreciate that the invention extends to such combinations of features and that these have not been recited in detail here in the interests of brevity.

In the context of describing the liquid composition described herein and in the appended claims, the singular forms “a”, “an”, and “the” include plural reference unless the context clearly dictates otherwise. Thus, for example, reference to “an excipient” is a reference to one or more excipients and equivalents thereof which will become apparent to those persons skilled in the art upon reading this disclosure and so forth.

The term “about” is used to indicate that a value includes the standard level of error for the device or method being employed to determine the value. As used herein, the term “about” means a slight variation of the value specified, for example, preferably within 10% of the value specified. The use of the term “or” in the claims is used to mean “and/or” unless explicitly indicated to refer to alternatives only or the alternatives are mutually exclusive, although the disclosure supports a definition that refers to only alternatives and to “and/or.” The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps; and also covers other unlisted steps.

The expressions “optional” or “optionally” means that the subsequently described event or circumstance may or may not occur, and that the description includes instances where the event occurs and instances where it does not.

The term “therapeutically effective amount” or “effective dose” as used herein refers to the amount or dose of pharmaceutically active ingredient that is sufficient to initiate therapeutic response in a patient.

The expression “pharmaceutically acceptable,” as used herein refers to an excipient having compatibility with the other ingredients of the formulation and not deleterious to the recipient thereof and also it means it is useful in preparing a pharmaceutical composition that is generally safe and non-toxic.

The term “composition” or “pharmaceutical composition” as used herein synonymously include liquid dosage forms such as solutions (aqueous and non-aqueous), suspensions, emulsions, syrups, elixirs, powder for solution, powder for suspension and the like meant for oral administration.

Oral liquids include, but are not limited to, solutions (both aqueous and nonaqueous), suspensions, emulsions, syrups, slurries, juices, elixirs, dispersions, powder for solution, powder for suspension and the like. It is envisioned that solution/suspensions are also included where certain components described herein are in a solution while other components are in a suspension. In some embodiments, the oral liquid formulation is a powder mixed with vehicle, before administration.

One aspect relates to an oral liquid composition comprising the powder for oral solution, prepared with a pharmaceutically acceptable liquid carrier. Another aspect relates to an oral liquid composition comprising the powder for oral suspension, prepared with a pharmaceutically acceptable liquid carrier.

The term “excipient” means a pharmacologically inactive component such as sweetening agent, complexing agent, stabilizing agent, viscosity modifiers, flavoring agent, antioxidant, preservative, buffering agent, suspending agent, solvent, co-solvent, chelating agent, suspension aids, etc., of a pharmaceutical product. The excipients that are useful in preparing a pharmaceutical composition are generally safe, non-toxic and are acceptable for human pharmaceutical use. Reference to an excipient includes both one and more than one of such excipients.

Angiotensin converting enzyme inhibitors include, but are not limited to, benazepril hydrochloride and similar salts; enalapril maleate and similar salts; quinapril hydrochloride and similar salts; moexipril hydrochloride and similar salts; lisinopril hydrochloride and similar salts; ramipril hydrochloride and similar salts; and perindopril and its salts. Typical breakdown products of angiotensin converting enzyme inhibitors include, but are not limited to, benazeprilat for benazepril drugs, enalaprilat and/or enalapril-diketopiperazine for enalapril drugs, quinaprilat and/or quinapril-diketopiperazine for quinapril drugs, perindoprilat for perindopril drugs and other breakdown products well-known to those of skill in the art.

Also, other angiotensin converting enzyme inhibitors may include alacipril, cilazapril, delapril, fosinopril, moveltipril, spirapril, ceronapril, imidapril, temocapril, trandolopril, utilbapril, zofenopril and the like, including their pharmaceutically acceptable salts. Captopril was the first angiotensin converting enzyme inhibitor drug to be developed for the treatment of hypertension.

Benazepril, benazeprilat, and their pharmaceutically acceptable salts are disclosed in U.S. Pat. No. 4,410,520, along with pharmaceutically acceptable dosage forms thereof, dosage ranges and suitable routes of administration therewith, and uses therefor, all of which are incorporated herein by reference.

Benazepril hydrochloride is a white to off-white crystalline powder, soluble (greater than 100 mg/mL) in water, in ethanol, and in methanol. Benazepril hydrochloride's chemical name is 3-[[1-(ethoxycarbonyl)-3-phenyl-(1S)-propyl]amino]-2,3,4,5-tetrahydro-2-oxo-1H-1-(3S)-benzazepine-1-acetic acid monohydrochloride. its structural formula is:

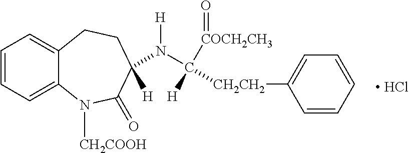

USFDA approved dihydropyridine calcium channel blockers include Clevidipine, Felodipine, Isradipine, Levamlodipine, Nicardipine, Nifedipine, Nimodipine and Nisoldipine.

As used herein, “amlodipine” refers to amlodipine base, its salt, or solvate or derivative or isomer or polymorph thereof. Suitable compounds include the free base, the organic and inorganic salts, isomers, isomer salts, solvates, polymorphs, complexes etc. Amlodipine itself is not satisfactory in photostability and preservation stability, and various salts are formed to improve them. Amlodipine salts include amlodipine benzoate, Amlodipine besylate, amlodipine tosylate, amlodipine mesylate, amlodipine succinate, amlodipine salicylate, amlodipine maleate, amlodipine acetate, amlodipine hydrochloride and the like.

In some embodiments, the amlodipine used in the formulations described herein is a pharmaceutically acceptable amlodipine salt. In some instances, the amlodipine salt is Amlodipine besylate, amlodipine benzoate. In other instances, the amlodipine salt is in the form of amlodipine naphthalene sulfonate. Amlodipine is referred to by the chemical name 3-ethyl-5-methyl (+)-2-[(2-aminoethoxy)methyl]-4-(2-chlorophenyl)-1,4-dihydro-6-methyl-3,5-pyridinedicarboxylate with a CAS No. of 88150-42-9.

Amlodipine besylate is a white to pale yellow crystalline powder with a molecular weight of 567.1. It is slightly soluble in water and sparingly soluble in ethanol. Its chemical name is (R,S) 3-ethyl-5-methyl-2-(2-aminoethoxymethyl)-4-(2-chlorophenyl)-1,4-dihydro-6-methyl3,5-pyridinedicarboxylate benzenesulfonate. its structural formula is:

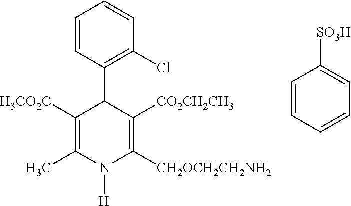

One embodiment of the present invention relates to stable oral liquid compositions comprising a therapeutically effective amount of angiotensin converting enzyme inhibitor and calcium channel blocker or their pharmaceutically acceptable salts thereof, a sweetening agent(s) and at least one pharmaceutically acceptable excipient.

Another embodiment of the present invention relates to stable liquid compositions for oral administration comprising a therapeutically effective amount of angiotensin converting enzyme inhibitor and calcium channel blocker or their pharmaceutically acceptable salts thereof and a sweetening agent(s) selected from glucose, fructose, sucrose, xylitol, tagatose, sucralose, maltitol, allulose, isomaltulose, isomalt, lactitol, sorbitol, erythritol, liquid glucose, compressible sugar, confectioner's sugar, dextrose, maltitol solution, a maltitol oligomer, maltose, mannitol, neohesperidin dihydrochalcone, thaumatin, trehalose, magnasweet 110, magnasweet 180, xylitol, sorbitan monoleate, trehalose, maltodextrin, polydextrose, glycerin, inulin, maltol, acesulfame and and its salts, alitame, aspartame, neotame, sodium cyclamate, saccharin and its salts, neohesperidin dihydrochalcone, stevioside, thaumatin, hydrogenated starch hydrolysates, maltitol syrup, high fructose corn syrup, sweet AM liquid/powder, prosweet, maltisweet, Sorbo®, invertose, rebalance M60 and X60, ora-sweet, ora-sweet-SF, monkfruit, flavored syrups, and the like or their combinations of two or more.

Another embodiment of the present invention relates to stable liquid composition for oral administration comprising a therapeutically effective amount of angiotensin converting enzyme inhibitor and calcium channel blocker and a sweetening agent(s) comprising an amount of about 0.5 to 75% w/w.

Another embodiment of the present invention relates to stable oral liquid compositions comprising a therapeutically effective amount of angiotensin converting enzyme inhibitor and calcium channel blocker or their pharmaceutically acceptable salts thereof, a complexing agent(s) and at least one pharmaceutically acceptable excipient.

Complexing agents may be used in enhancing the properties of the presently disclosed composition. Many drugs have poor water solubility, which can limit their effectiveness. Complexing agents can form inclusion complexes with these drugs, significantly improving their solubility and, consequently, their bioavailability. Complexing agents can stabilize drugs by protecting them from degradation. For example, they can prevent oxidation or hydrolysis, thereby extending the shelf life of the drug. By forming complexes with metal ions or other potentially harmful substances, complexing agents can reduce the toxicity of certain drugs. Complexing agents can improve the delivery of drugs to their target sites in the body. This can be particularly useful for drugs that need to cross biological membranes or be delivered to specific tissues.

Another embodiment of the present invention relates to stable liquid compositions for oral administration comprising a therapeutically effective amount of angiotensin converting enzyme inhibitor and calcium channel blocker or their pharmaceutically acceptable salts thereof and a complexing agent(s) selected from ethylenediaminetetraacetic acid, cyclodextrins (unmodified cyclodextrins such as α-cyclodextrin, β-cyclodextrin and γ-cyclodextrin; substituted cyclodextrins such as partly substituted hydroxyalkylcyclodextrins, such as hydroxypropylcyclodextrin or dihydroxypropylcyclodextrin, partly substituted carboxyalkylcyclodextrins, such as carboxymethylcyclodextrin, alkali metal salts of partly substituted carboxyalkylcyclodextrins or partly substituted water-soluble alkylcyclodextrins, such as C1 to C4 alkylcyclodextrins; hydroxypropyl-β-cyclodextrins, sulfobutylether-β-cyclodextrins; methylated β-cyclodextrins such as heptakis (2,6-di-O-methyl)-β-cyclodextrin), RAMEB (statistically methylated or randomly-methylated-β-cyclodextrin β-cyclodextrin), TRIMEB (heptakis (2,3,6-tri-O-methyl)-β-cyclodextrin) and CRYSMEB (weakly methylated β-cyclodextrin) and the like), phosphates, zeolites, ammonia, EDTA and their salts, tartaric acid, citric acid, polyvinylpyrrolidone, polyethylene glycol and its derivatives, cross linked polymers such as carbomers, crosslinked polyvinylpyrrolidone, crosslinked sodium carboxyl methyl cellulose, polyacrylamide, cross linked hydroxyl propyl methyl cellulose, polyethylene glycol diacrylate, cross linked chitosan and the like or their combinations of two or more.

Another embodiment of the present invention relates to stable liquid composition for oral administration comprising a therapeutically effective amount of angiotensin converting enzyme inhibitor and calcium channel blocker and a complexing agent(s) comprising in amount of about 0.5 to 50% w/w.

Another embodiment of the present invention relates to stable oral liquid compositions comprising a therapeutically effective amount of angiotensin converting enzyme inhibitor and calcium channel blocker or their pharmaceutically acceptable salts thereof, a stabilizing agent(s) and at least one pharmaceutically acceptable excipient.

A stabilizing agent may be used to maintain the physical and chemical properties of a material, preventing degradation and extending its useful life. Stabilizing agents can protect drugs from degradation, improving their shelf life and effectiveness.

Another embodiment of the present invention relates to stable liquid compositions for oral administration comprising a therapeutically effective amount of angiotensin converting enzyme inhibitor and calcium channel blocker or their pharmaceutically acceptable salts thereof and a stabilizing agent(s) selected from cyclodextrins, polyvinylpyrrolidone, sodium alginate, gelatin, silicon dioxide and the like or their combinations of two or more.

Another embodiment of the present invention relates to stable liquid composition for oral administration comprising a therapeutically effective amount of angiotensin converting enzyme inhibitor and calcium channel blocker and a stabilizing agent(s) comprising in amount of about 0.5 to 50% w/w.

The complexing agent, solubilizing agent, stabilizing agent and chelating agent of the present disclosure are interlinked and the drug components complexed with these agents enhance solubility and stability to form a stable oral liquid composition of the invention.

Viscosity modifiers, also known as viscosity thickeners, viscosity enhancers or viscosity-increasing agents, may be used to improve the consistency and stability of liquid formulations.

Another embodiment of the present invention relates to stable liquid compositions for oral administration comprising a therapeutically effective amount of angiotensin converting enzyme inhibitor and calcium channel blocker or their pharmaceutically acceptable salts thereof and a viscosity modifier(s) selected from hydrocolloid gums, cellulose derivatives, hydroxyethylcellulose, methylcellulose, carboxymethylcellulose, carbomers, polyvinyl alcohols, silicon dioxide and the like or their combinations of two or more.

Another embodiment of the present invention relates to stable liquid composition for oral administration comprising a therapeutically effective amount of angiotensin converting enzyme inhibitor and calcium channel blocker and a viscosity modifier(s) comprising an amount of about 0.05 to 7% w/w.

Another embodiment of the present invention relates to stable oral liquid compositions comprising a therapeutically effective amount of angiotensin converting enzyme inhibitor and calcium channel blocker or their pharmaceutically acceptable salts thereof, a flavoring agent(s) and at least one pharmaceutically acceptable excipient.

Another embodiment of the present invention relates to stable liquid compositions for oral administration comprising a therapeutically effective amount of angiotensin converting enzyme inhibitor and calcium channel blocker or their pharmaceutically acceptable salts thereof and a flavoring agent(s) selected from almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, wintergreen; cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof; vanilla, citrus oil, fruit essences, including apple, banana, pear, peach, strawberry, raspberry, cherry, plums pineapple, apricot, peppermint, tutti frutti, mixed berry, and so forth and the like or any combinations thereof. Solid forms, such as spray dried forms of flavoring agents, may also be useful in the liquid dosage forms disclosed herein.

Another embodiment of the present invention relates to stable liquid composition for oral administration comprising a therapeutically effective amount of angiotensin converting enzyme inhibitor and calcium channel blocker and a flavoring agent comprising an amount of about 0.05 to 3% w/w.

Another embodiment of the present invention relates to stable oral liquid compositions comprising a therapeutically effective amount of angiotensin converting enzyme inhibitor and calcium channel blocker or their pharmaceutically acceptable salts thereof, one or more antioxidants/preservatives and at least one pharmaceutically acceptable excipient.

Another embodiment of the present invention relates to stable liquid compositions for oral administration comprising a therapeutically effective amount of angiotensin converting enzyme inhibitor and calcium channel blocker or their pharmaceutically acceptable salts thereof and an antioxidants/preservatives selected from ascorbic acid, ascorbyl palmitate, butylated hydroxyanisole, butylated hydroxytoluene, citric acid, EDTA and its salts, erythorbic acid, fumaric acid, malic acid, sorbic acid, propyl gallate, ethyl oleate, methionine, monothioglycerol, thymol, tocopherols, vitamin E, vitamin E polyethylene glycol succinate, sodium ascorbate, sodium bisulfate, sodium metabisulfite, sodium sulfite, parabens (methylparaben, ethylparaben, propylparaben, butylparaben, isobutylparaben and the like) and/or their salts, benzoic acid, sodium benzoate, potassium sorbate, vanillin, benzyl alcohol, boric acid, calcium acetate, bentonite, cetrimide, chlorhexidine, cetylpyridinium chloride, cresol, chlorbutanol, magnesium trisilicate and the like or their combinations of two or more.

Another embodiment of the present invention relates to stable liquid composition for oral administration comprising a therapeutically effective amount of angiotensin converting enzyme inhibitor and calcium channel blocker and an antioxidant/preservative comprising an amount of about 0.01 to 2.20% w/w.

Another embodiment of the present invention relates to stable oral liquid compositions comprising a therapeutically effective amount of angiotensin converting enzyme inhibitor and calcium channel blocker or their pharmaceutically acceptable salts thereof, one or more buffering agents and at least one pharmaceutically acceptable excipient.

Another embodiment of the present invention relates to stable liquid compositions for oral administration comprising a therapeutically effective amount of angiotensin converting enzyme inhibitor and calcium channel blocker or their pharmaceutically acceptable salts thereof and a buffering agent(s) selected from sodium bicarbonate, potassium bicarbonate, magnesium hydroxide, magnesium lactate, magnesium gluconate, aluminum hydroxide, aluminum glycinate, citric acid, fumaric acid, tartaric acid, maleic acid, lactic acid, hydrochloric acid, phosphoric acid, glycine, glacial acetic acid, sodium acetate trihydrate, trisodium citrate, potassium chloride, hydroxymethyl aminomethane, sodium hydroxide, sodium citrate, sodium tartrate, sodium acetate, sodium carbonate, sodium phosphate monobasic, potassium phosphate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium carbonate, magnesium silicate, calcium acetate, calcium glycerophosphate, calcium chloride, calcium hydroxide, calcium lactate, calcium carbonate, calcium bicarbonate, and the like or their combinations of two or more.

Another embodiment of the present invention relates to stable liquid composition for oral administration comprising a therapeutically effective amount of angiotensin converting enzyme inhibitor and calcium channel blocker and a buffering agent comprising an amount of about 0.05 to 10% w/w.

Another embodiment of the present invention relates to stable oral liquid compositions comprising a therapeutically effective amount of angiotensin converting enzyme inhibitor and calcium channel blocker or their pharmaceutically acceptable salts thereof, a suspending agent(s) and at least one pharmaceutically acceptable excipient.

Suspending agents may be used in the present pharmaceutical formulations to ensure that solid particles remain uniformly distributed within a liquid medium, maintaining the physical stability of the product. These agents are selected based on their ability to maintain the stability of the suspension, prevent sedimentation, and ensure the uniform distribution of the active pharmaceutical ingredient(s).

Another embodiment of the present invention relates to stable liquid compositions for oral administration comprising a therapeutically effective amount of angiotensin converting enzyme inhibitor and calcium channel blocker or their pharmaceutically acceptable salts thereof and a suspending agent(s) selected from methylcellulose, microcrystalline cellulose, sodium carboxymethylcellulose, acacia, xanthan gum, gellan gum, bentonite and the like or their combinations of two or more.

Another embodiment of the present invention relates to stable liquid composition for oral administration comprising a therapeutically effective amount of angiotensin converting enzyme inhibitor and calcium channel blocker and a suspending agent(s) comprising an amount of about 0.05 to 20% w/w.

Another embodiment of the present invention relates to stable oral liquid compositions comprising a therapeutically effective amount of angiotensin converting enzyme inhibitor and calcium channel blocker or their pharmaceutically acceptable salts thereof, a viscosity modifier(s) and at least one pharmaceutically acceptable excipient.

Suitable suspension aids/suspension stabilizer/gelling agent/thickeners include, but are not limited to, dextrin, cellulose derivatives (carboxymethylcellulose and its salts, ethylcellulose, hydroxypropyl methylcellulose, hydroxyethyl cellulose, methylcellulose, hypromellose, and the like) starches, pectin, polyethylene glycol, polyethylene oxide, trehalose, certain silicates (magnesium aluminum silicate, aluminum silicate, etc. such as Veegum, Bentonite, and Kaolin) and certain gums (xanthan gum, guar gum, gum arabic, locust bean gum, etc.), silicon dioxide, microcrystalline cellulose, polyvinylpyrrolidone, 3-butoxy-2-hydroxypropylhydroxyethylcellulose, acrylamide homo- and copolymers, acrylic acid homo- and copolymer, alginates, carboxymethylhydroxyethylcellulose, carboxy-vinyl copolymers, cellulose, microcrystalline cellulose (of different grades), hydrophobically modified hydroxyethylcellulose, hydroxypropyl guar, hydroxypropyl methylcellulose, hydroxypropylcellulose, partially and fully hydrolyzed polyvinyl alcohols, partially neutralized polyacrylic acid, polyalkylene glycol, polysaccharide gums, polyvinylpyrrolidones, starches, vinylpyrrolidone homo- and copolymers, water-soluble cellulose ethers, bentonite, laponites, kaolinite, dickite, nacrite, pyrophylite, talc, vermiculite, sauconite, saponite, nontronite, montmorillonite, organically modified montmorillonite clays and the like or their combinations of two or more in an amount of about 0.05 to 30% w/w.

Examples of a solvent/vehicle include, but are not limited to, purified water, alcohols, isopropyl alcohol and the like or their combinations of two or more in an amount to produce 100% for oral administration.

Examples of a co-solvent include, but are not limited to, glycerin, an alcohol (short-chain substituted or non-substituted alcohols such as ethanol, isopropanol, or propanol and the like), a glycol (propylene glycol, polyethylene glycol and the like), and the like or their combinations of two or more in an amount to produce 100% for oral administration.

Examples of a chelating agent include, but are not limited to, ethylenediamine tetraacetic acid, ammonium citrate, benzotriazole, calcium phytate, nitrilotriacetic acid, n-hydroxyethylethylenediaminetriacetic acid, and the like or their combinations of two or more in an amount of about 0.01 to 3% w/w.

Examples of additional excipients include, but are not limited to, base pellets (mannitol and sugar spheres and the like); coating agents (hydroxypropyl methyl cellulose, hydroxy propyl cellulose, polyvinyl alcohol, phthalate, and hydroxyl ethyl cellulose, eudragits and the like); talc, resin and their combination thereof in pharmaceutically acceptable quantity.

Additional excipients are contemplated in the oral liquid formulation embodiments. These additional excipients are selected based on function and compatibility with the oral liquid formulations described herein and may be found, for example in Remington: The Science and Practice of Pharmacy, Nineteenth Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N. Y.: Marcel Decker 1980); and Pharmaceutical Dosage Forms and Drug Delivery Systems, Seventh Ed (Lippincott Williams & Wilkins 1999), herein incorporated by reference in their entirety.

One aspect relates to a stable oral liquid formulation comprising angiotensin converting enzyme inhibitor, calcium channel blocker and one or more pharmaceutically acceptable excipients for the treatment of cardiovascular and other related diseases including hypertension, coronary artery disease, heart failure and acute myocardial infarction.

Yet another aspect relates to a stable, oral liquid pharmaceutical formulation comprising;
(i) benazepril or a pharmaceutically acceptable salt or solvate thereof;
(ii) amlodipine or a pharmaceutically acceptable salt or solvate thereof; and
(iii) a sweetening agent.

Another aspect relates to a stable, oral liquid pharmaceutical formulation comprising;
(i) benazepril or a pharmaceutically acceptable salt or solvate thereof;
(ii) amlodipine or a pharmaceutically acceptable salt or solvate thereof; and
(iii) a complexing agent.

Another aspect relates to a stable, oral liquid pharmaceutical formulation comprising;
(i) benazepril or a pharmaceutically acceptable salt or solvate thereof;
(ii) amlodipine or a pharmaceutically acceptable salt or solvate thereof; and
(iii) a stabilizing agent.

Another aspect relates to a stable oral liquid pharmaceutical formulation comprising;
(i) benazepril or a pharmaceutically acceptable salt or solvate thereof;
(ii) amlodipine or a pharmaceutically acceptable salt or solvate thereof; and
(iii) a viscosity modifier.

Another aspect relates to a stable, oral liquid pharmaceutical formulation comprising;
(i) benazepril or a pharmaceutically acceptable salt or solvate thereof;
(ii) amlodipine or a pharmaceutically acceptable salt or solvate thereof;
(iii) a sweetening agent; and
(iv) a complexing agent.

Another aspect relates to a stable oral liquid pharmaceutical formulation comprising;
(i) benazepril or a pharmaceutically acceptable salt or solvate thereof;
(ii) amlodipine or a pharmaceutically acceptable salt or solvate thereof;
(iii) a sweetening agent;
(iv) a complexing agent; and
(v) a viscosity modifier.

Another aspect relates to a stable oral liquid pharmaceutical formulation comprising;
(i) benazepril or a pharmaceutically acceptable salt or solvate thereof;
(ii) amlodipine or a pharmaceutically acceptable salt or solvate thereof;
(iii) a sweetening agent;
(iv) a complexing agent;
(v) a stabilizing agent;
(vi) a viscosity modifier; and
(vii) one or more pharmaceutical acceptable excipients include but not limited to a flavoring agent, a preservative/antioxidant, a buffering agent, a suspending agent, a solvent/vehicle, a cosolvent, a chelating agent, and the like.

One embodiment of the present invention of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof; and
(iii) about 0.5% to about 75% w/w of a sweetening agent.

Another embodiment of the present invention, of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof; and
(iii) about 0.5% to about 50% w/w of a complexing agent.

Another embodiment of the present invention, of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof; and
(iii) about 0.5% to about 50% w/w of a stabilizing agent.

Another embodiment of the present invention, of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof; and
(iii) about 0.05% to about 7% w/w of a viscosity modifier.

Another embodiment of the present invention, of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof; and
(iii) about 0.5% to about 75% w/w of a sweetening agent; and
(iv) about 0.5% to about 50% w/w of a complexing agent.

Another embodiment of the present invention, of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof;
(iii) about 0.5% to about 75% w/w of a sweetening agent;
(iv) about 0.5% to about 50% w/w of a complexing agent; and
(v) about 0.05% to about 7% w/w of a viscosity modifier.

Another embodiment of the present invention, of the stable oral liquid formulation comprises:
(i) about 0.3% to about 7% w/w of benazepril or a pharmaceutically acceptable salt or solvate thereof;
(ii) about 0.05% to about 2% w/w of amlodipine or a pharmaceutically acceptable salt or solvate thereof;
(iii) about 0.5% to about 75% w/w of a sweetening agent;
(iv) about 0.5% to about 50% w/w of a complexing agent;
(v) about 0.05% to about 7% w/w of a viscosity modifier; and
(vi) one or more pharmaceutical acceptable excipients include but not limited to flavoring agent (about 0.05-3% w/w), preservative/antioxidant (about 0.01-2.20% w/w), buffering agent (about 0.05-10% w/w), suspending agent (about 0.05-20% w/w), solvent/vehicle (q.s.), cosolvent (q.s.), chelating agent (about 0.01-3% w/w), and the like.

In another embodiment of the present invention, the oral liquid formulation has a pH between about 3 and about 7.5 and the stability of the formulation was evaluated.

Also provided herein are methods of treating hypertension comprising administering to a patient in need thereof a benazepril and amlodipine oral liquid formulation described herein. In some embodiments, the hypertension is primary (essential) hypertension. In some embodiments, the hypertension is secondary hypertension. In some embodiments, the subject with hypertension has blood pressure values greater than or equal to 140/90 mmm Hg.

Another aspect disclosed herein relates to a method for the symptomatic treatment of a chronic stable angina in a patient in need thereof, which comprises administering to said patient a therapeutically effective amount of any one of the formulations disclosed herein.

Yet another aspect disclosed herein relates to a method for the treatment of confirmed or suspected vasospastic angina in a patient in need thereof, which comprises administering to said patient a therapeutically effective amount of any one of the formulations disclosed herein.

### EXAMPLES

The following examples further describe and demonstrate particular embodiments within the scope of the present invention. The examples are given solely for illustration and are not to be construed as limitations as many variations are possible without departing from the spirit and scope of the invention.

### A. General Compositions of the Invention

| Formulation 1 | % w/w | Formulation 2 | % w/w |
|---|---|---|---|
| Benazepril hydrochloride | 0.3-7 | Benazepril hydrochloride | 0.3-7 |
| Amlodipine besylate | 0.05-2 | Amlodipine besylate | 0.05-2 |
| Sweetening agent | 0.5-75 | Complexing agent | 0.5-50 |
| Vehicle/solvent | Q.S. | Vehicle/solvent | Q.S. |
| and/or cosolvent | | and/or cosolvent | |
| Formulation 3 | % w/w | Formulation 4 | % w/w |
| Benazepril hydrochloride | 0.3-7 | Benazepril hydrochloride | 0.3-7 |
| Amlodipine besylate | 0.05-2 | Amlodipine besylate | 0.05-2 |
| Stabilizing agent | 0.5-50 | Viscosity modifiers | 0.05-7 |
| Vehicle/solvent | Q.S. | Vehicle/solvent | Q.S. |
| and/or cosolvent | | and/or cosolvent | |
| Formulation 5 | % w/w | Formulation 6 | % w/w |
| Benazepril hydrochloride | 0.3-7 | Benazepril hydrochloride | 0.3-7 |
| Amlodipine besylate | 0.05-2 | Amlodipine besylate | 0.05-2 |
| Sweetening agent | 0.5-75 | Sweetening agent | 0.5-75 |
| Complexing agent | 0.5-50 | Complexing agent | 0.5-50 |
| and/or Optional | | and/or Optional | |
| Stabilizing agent | | Stabilizing agent | |
| Vehicle/solvent | Q.S. | Viscosity modifiers | 0.05-7 |
| and/or cosolvent | | Vehicle/solvent | Q.S. |
| | | and/or cosolvent | |

### B. Specific Composition of the Invention

| Ingredients | % w/w |
|---|---|
| Benazepril hydrochloride | 0.3-7 |
| Amlodipine besylate | 0.05-2 |
| Sweetening agent | 0.5-75 |
| Complexing agent | 0.5-50 |
| Optional Stabilizing agent | 0.5-50 |
| Viscosity modifiers | 0.05-7 |
| Excipient* | Q.S. |

*one or more selected from the group comprises flavoring agent (about 0.05-3% w/w), preservative/antioxidant (about 0.01-2.20% w/w), buffering agent (about 0.05-10% w/w), suspending agent (about 0.05-20% w/w), solvent/vehicle (q.s.), cosolvent (q.s.), chelating agent (about 0.01-3% w/w), and the like and their combinations thereof.

### Example 1

| Ingredients | Function | % w/w |
|---|---|---|
| Benazepril hydrochloride | Active pharmaceutical ingredient | 0.3-7 |
| Amlodipine besylate | Active pharmaceutical ingredient | 0.05-2 |
| Maltitol/Mannitol/Sucralose | Sweetening agent | 0.5-75 |
| Hydroxy β-cyclodextrin | Complexing agent | 0.5-50 |
| Purified water and/or | Vehicle/solvent | q.s. |
| isopropyl alcohol | | |

### Example 2

| Ingredients | Function | % w/w |
|---|---|---|
| Benazepril hydrochloride | Active pharmaceutical | 0.3-7 |
| | ingredient | |
| Amlodipine besylate | Active pharmaceutical | 0.05-2 |
| | ingredient | |
| Maltitol/Mannitol/Sucralose | Sweetening agent | 0.5-75 |
| Hydroxy β-cyclodextrin | Complexing agent | 0.5-50 |
| Peppermint flavor | Flavoring agent | 0.05-3 |
| Butylated hydroxy anisole | Antioxidant/Preservative | 0.01-2.20 |
| and/or sodium benzoate | | |
| Citric acid monohydrate | Buffering agent | 0.05-10 |
| and sodium citrate | | |

### Example 3

| Ingredients | Function | % w/w |
|---|---|---|
| Benazepril hydrochloride | Active pharmaceutical | 0.3-7 |
| | ingredient | |
| Amlodipine besylate | Active pharmaceutical | 0.05-2 |
| | ingredient | |
| Maltitol/Mannitol/Sucralose | Sweetening agent | 0.5-75 |
| Hydroxy β-cyclodextrin | Complexing agent | 0.5-50 |
| Peppermint flavor | Flavoring agent | 0.05-3 |
| Butylated hydroxy anisole | Antioxidant/Preservative | 0.01-2.20 |
| and/or sodium benzoate | | |
| Citric acid monohydrate | Buffering agent | 0.05-10 |
| and sodium citrate | | |
| Purified water and/or | Vehicle/Solvent | q.s. |
| isopropyl alcohol | | |

The above three examples may also include a stabilizing agent and/or viscosity modifier. In embodiments, the stabilizing agent may be povidone in an amount of about 0.5 to about 50% w/w and the viscosity modifier may be hydroxyethyl cellulose in an amount of from about 0.05 to about 7% w/w.

### Example 4: Oral Solution

| Ingredients | Function | % w/w |
|---|---|---|
| Benazepril hydrochloride | Active pharmaceutical | 0.3-0.9 |
| | ingredient | |
| Amlodipine besylate | Active pharmaceutical | 0.05-0.3 |
| | ingredient | |
| Maltitol/Mannitol/Sucralose | Sweetening agent | 0.5-60 |
| Hydroxy β-cyclodextrin | Complexing agent | 0.5-50 |
| Povidone | Stabilizing agent | 0.5-50 |
| Hydroxyethyl cellulose | Viscosity modifier | 0.05-7 |
| Peppermint flavor | Flavouring agent | 0.05-3 |
| Butylated hydroxy anisole | Antioxidant | 0.01-0.15 |
| Sodium benzoate | Antimicrobial Preservative | 0.05-2 |
| Citric acid monohydrate | Buffering agent | 0.05-5 |
| Sodium citrate | Buffering agent | 0.1-5 |
| Disodium EDTA | Chelating agent | 0.01-3 |
| Purified water | Solvent/vehicle | q.s |
| Glycerin | Cosolvent | 10.0-65 |
| Propylene glycol | Cosolvent | 5.0-30 |

### General Manufacturing Process for Oral Solution

An oral solution comprising amlodipine besylate and benazepril hydrochloride may be prepared according to the following representative procedure.
1. Preparation of Cyclodextrin Solution: Hydroxy β-cyclodextrin is added to purified water in a suitable vessel and mixed until completely dissolved to obtain a clear solution.
2. Incorporation of Amlodipine Besylate: Amlodipine besylate is added to the solution obtained in Step 1 and mixed until complete dissolution is achieved, resulting in a clear solution.
3. Addition of Preservative: Sodium benzoate is added to the mixture from Step 2 and stirred until completely dissolved to obtain a clear solution.
4. Preparation of Antioxidant Solution: Butylated hydroxyanisole is dissolved in propylene glycol in a separate vessel with mixing until a clear solution is obtained.
5. Combination of Solutions: The antioxidant solution of Step 4 solution is added to the solution obtained in Step 3 under continuous stirring until uniform and clear.
6. Preparation of Benazepril Hydrochloride Solution: Benazepril hydrochloride is dissolved in purified water in a separate vessel with mixing until a clear solution is obtained.
7. Preparation of Thickening Agent Solution: Hydroxyethyl cellulose is added to refined glycerin in a separate vessel and mixed until completely dissolved to obtain a clear solution.
8. Combination with Thickening Agent Solution: The hydroxyethyl cellulose solution from Step 7 is added to the mixture from Step 5 under stirring until a uniform and clear solution is obtained.
9. Addition of Sweeteners: Maltitol, Mannitol, or sucralose is added to the mixture from Step 8 under continuous stirring until completely dissolved.
10. Incorporation of Benazepril Hydrochloride Solution: The benazepril hydrochloride solution from Step 6 is added to the mixture from Step 9 with stirring until a clear and homogenous solution is obtained.
11. Addition of Buffering Agents and Flavoring: Citric acid monohydrate, sodium citrate, disodium EDTA and peppermint flavor are added to the mixture of Step 10 with stirring until completely mixed to form a clear solution.
12. Adjustment of Final Volume: The final volume is adjusted with purified water to yield the desired concentration.
13. Filling: The resultant clear oral solution is filled into suitable bottles under standard conditions.

### Example 5: Powder for Oral Solution

| Ingredients | Function | % w/w |
|---|---|---|
| Benazepril hydrochloride | Active pharmaceutical ingredient | 0.5-7 |
| Amlodipine besylate | Active pharmaceutical ingredient | 0.5-2 |
| Maltitol/Mannitol/Sucralose | Sweetening agent | 10-75 |
| Hydroxy β-cyclodextrin | Complexing agent | 0.5-50 |
| Peppermint flavor | Flavouring agent | 0.05-3 |
| Butylated hydroxy anisole | Antioxidant | 0.01-0.2 |
| Sodium benzoate | Antimicrobial Preservative | 0.05-2 |
| Citric acid monohydrate | Buffering agent | 0.05-5 |
| Sodium citrate | Buffering agent | 0.1-5 |
| Disodium EDTA | Chelating agent | 0.01-3 |
| Hydroxypropyl | Coating agent | 1-15 |
| Methylcellulose | | |
| Isopropyl alcohol/ | Solvent/Vehicle | q.s |
| Purified water | | |

The above example may also include a stabilizing agent, and/or viscosity modifier, and/or a sweetening agent and/or a coating agent. In embodiments, the stabilizing agent may be povidone in an amount of about 0.5 to about 50% w/w and the viscosity modifier may be hydroxyethyl cellulose in an amount of from about 0.05 to about 7% w/w. In embodiments, the sweetening agent may be mannitol or sugar spheres in an amount of from about 1-15% w/w and the coating agent may be hydroxypropyl cellulose in an amount of from about 0.1-5% w/w.

### General Manufacturing Process of Powder for Oral Solution

### Preparation of Amlodipine Part:

1. Sifting of Excipients: Maltitol, mannitol, and/or sucralose are passed through a suitable sieve and transferred to a rapid mixing granulator (RMG).
2. Preparation of Binder Solution: A measured quantity of isopropyl alcohol/purified water mixture is charged into a stainless steel (SS) vessel. Butylated hydroxy anisole is added under stirring until completely dissolved. Hydroxy β-cyclodextrin or povidone is then added and mixed until a lump-free dispersion is obtained.
3. Addition of Active Ingredient: Amlodipine besylate is added to the dispersion obtained from Step 2 dispersion and stirred until a lump free dispersion is formed.
4. Dry Mixing: The sifted materials from Step 1 are mixed in the RMG for approximately 10 minutes with the impeller ON and the chopper OFF.
5. Wet Granulation: The binder solution prepared in Step 3 is added to the mixture of Step 4 with the impeller ON and chopper OFF. Mixing continues until uniform wetting is achieved.
6. Kneading: The resulting wet mass is kneaded with both impeller ON and chopper ON until suitable granule formation is observed.
7. Adjustment of Moisture (if required): If necessary, an additional quantity of the isopropyl alcohol/purified water mixture may be added with the impeller ON and chopper OFF, followed by kneading with both impeller and chopper ON to achieve a uniform granulate. Drying: The wet granules are transferred to a fluid bed dryer (FBD) and dried at a controlled temperature until a loss on drying (LOD) not more than 2.0% w/w is attained.
8. Milling and Sieving: The dried granules obtained from Step 8 are milled and passed through a suitable sieve to obtain uniform particle size, and the milled granules are collected for further processing.

### Preparation of Benazepril Hydrochloride Part—Processing by Direct Blending:

1. Benazepril hydrochloride, maltitol/mannitol/sucralose, hydroxyethyl cellulose, sodium benzoate, disodium edetate, citric acid monohydrate, sodium citrate and peppermint flavor are sifted through a suitable sieve.
2. The sifted materials are blended for approximately 10 minutes to obtain a uniform mixture.

### Preparation of Benazepril Hydrochloride Part—Processing by Top Spray Technique:

The benazepril hydrochloride part was prepared by a top spray coating process as described below.
1. Benazepril hydrochloride and maltitol/mannitol/sucralose are sifted through a suitable sieve.
2. A weighed quantity of isopropyl alcohol/Purified water mixture is placed in a SS vessel. Opadry Clear (hydroxypropyl methylcellulose-based coating material) is slowly dispersed under stirring until a uniform solution is obtained.
3. The sifted materials from Step 1 are loaded into a fluid bed processer and pre-warmed to a product temperature of 30° C. for 10±5 mins.
4. The pre-warmed materials are coated with the prepared coating solution via a top-spray technique until the desired weight gain is achieved.
5. The coated granules are sifted and collected.
6. Sodium benzoate, citric acid monohydrate, sodium citrate, disodium EDTA, hydroxyethyl cellulose and peppermint flavor are sifted separately.
7. The coated granules from Step 5 are transferred to a blender, the sifted ingredients from Step 6 are added, and the mixture is blended to obtain a homogenous composition.
8. Final Assembly and Filling: Fill the granules from the Amlodipine part granules and Benazepril part are combined and filled granules into individual a sachets or in a suitable glass, or PET, or polypropylene (PP) bottles under controlled environmental conditions to yield the final powder for oral solution.

### Example 6: Oral Suspension

| Ingredients | Function | % w/w |
|---|---|---|
| Benazepril hydrochloride | Active pharmaceutical | 0.3-0.9 |
| | ingredient | |
| Amlodipine besylate | Active pharmaceutical | 0.05-0.3 |
| | ingredient | |
| Maltitol/Mannitol/Sucralose | Sweetening agent | 0.5-60 |
| Hydroxy β-cyclodextrin | Complexing agent | 0.5-30 |
| Povidone | Stabilizing agent | 0.5-30 |
| Hydroxyethyl cellulose | Viscosity modifier | 0.05-7 |
| Peppermint flavor | Flavouring agent | 0.05-3 |
| Butylated hydroxy anisole | Antioxidant | 0.01-0.15 |
| Sodium benzoate | Antimicrobial preservative | 0.05-2 |
| Citric acid monohydrate | Buffering agent | 0.05-5 |
| Sodium citrate | Buffering agent | 0.1-5 |
| Disodium EDTA | Chelating agent | 0.01-3 |
| Purified water/Isopropyl alcohol | Solvent/vehicle | q.s |
| Glycerin | Cosolvent | 10.0-65 |
| Propylene glycol | Cosolvent | 5.0-30 |
| Microcrystalline cellulose | Suspending agent | 0.2-15 |
| Carboxymethylcellulose sodium | Suspending agent/ | 0.05-10 |
| | Viscosity modifier | |
| Crospovidone/ | Anticaking agent | 0.05-10 |
| Colloidal silicon dioxide | | |

### General Manufacturing Process for Oral Suspension

Example 6 was prepared according to the general procedure described in Example 1, except that in Step 11, crospovidone and/or colloidal silicon dioxide were additionally incorporated into the solution of Step 10 under continuous stirring. The resulting mixture was stirred until a uniform dispersion was obtained, thereby forming an oral suspension instead of a clear oral solution. All other steps and processing parameters were substantially the same as those described in Example 1.

### Example 7: Powder/Pellet for Oral Suspension

| Ingredients | Function | % w/w |
|---|---|---|
| Benazepril hydrochloride | Active pharmaceutical | 0.5-7 |
| | ingredient | |
| Amlodipine besylate | Active pharmaceutical | 0.5-2 |
| | ingredient | |
| Maltitol/Mannitol/Sucralose | Sweetening agent | 10-75 |
| Hydroxy β-cyclodextrin | Complexing agent | 0.5-40 |
| Povidone (optional) | Stabilizing agent | 0.5-40 |
| Hydroxyethyl cellulose (optional) | Viscosity modifier | 0.05-7 |
| Peppermint flavor | Flavouring agent | 0.05-3 |
| Butylated hydroxy anisole | Antioxidant | 0.01-0.2 |
| Sodium benzoate | Antimicrobial | 0.05-2 |
| | preservative | |
| Citric acid monohydrate | Buffering agent | 0.05-5 |
| Sodium citrate | Buffering agent | 0.1-5 |
| Disodium EDTA | Chelating agent | 0.01-3 |
| Mannitol/sugar spheres | Base pellets | 1-15 |
| Hydroxypropyl Methylcellulose | Coating agent | 1-15 |
| Microcrystalline cellulose | Suspending agent | 0.2-15 |
| Carboxymethylcellulose | Suspending agent/ | 0.05-10 |
| sodium | Viscosity modifier | |
| Crospovidone/ | Anticaking agent | 0.05-10 |
| Colloidal silicon dioxide | | |
| Xanthan gum | Gelling agent/Suspending | 0.05-7 |
| | agent/Viscosity modifier | |
| Eudragit E 100 Polymer (optional) | Coating polymer | 1-15 |
| Resin (Kyron complex) (optional) | Complexing agent | 1-15 |
| Hydroxypropyl cellulose | Coating agent | 0.5-5 |
| (optional) | | |
| Talc (optional) | Anticaking agent/ | 0.05-5 |
| | Lubricant | |
| Isopropyl alcohol/Purified water | Solvent/Vehicle | q.s |

### General Manufacturing Process of Powder/Pellet for Oral Suspension

### Preparation of Amlodipine Part:

Example 7 was carried out in accordance with the general procedure described in Example 2 for the preparation of the amlodipine part. All processing steps, materials, and parameters were substantially the same as those set forth in Example 2, except where otherwise indicated. The resulting granules were uniform, free-flowing, and suitable for subsequent blending or formulation with other active components.

### Preparation of Benazepril Hydrochloride Part—Processing by Direct Blending:

Example 7 was conducted in accordance with the general procedure described in Example 2 for the preparation of the Benazepril hydrochloride part. All process steps, materials, and conditions were substantially the same as in Example 2, except that xanthan gum, microcrystalline cellulose, sodium carboxymethyl cellulose, and crospovidone and/or colloidal silicon dioxide were additionally incorporated into the formulation. These excipients were blended with the Benazepril hydrochloride mixture prior to the final blending step to enhance suspension stability and uniformity of the resulting granules.

### Preparation of Benazepril Hydrochloride Part—Processing by Top Spray Technique:

Example 7 was carried out in accordance with the general procedure described in Example 2 for the preparation of the Benazepril hydrochloride Part B using the spray-coating technique. All processing steps, materials, and conditions were substantially the same as those in Example 2, except that microcrystalline cellulose, sodium carboxymethyl cellulose, xanthan gum, and crospovidone and/or colloidal silicon dioxide were additionally incorporated into the formulation. These excipients were introduced during the blending stage following coating and drying of the granules to improve flow characteristics, suspension stability, and overall content uniformity of the final product.

### Preparation of Benazepril Hydrochloride Part—Processing by Wurster Technique:

Example 7 was carried out in accordance with the general procedure described in Example 2 for the preparation of the Benazepril hydrochloride Part C using the Wurster coating technique. All materials, process parameters, and operational steps were substantially the same as in Example 2, except that in step 8, microcrystalline cellulose, sodium carboxymethyl cellulose, xanthan gum, and crospovidone and/or colloidal silicon dioxide were added without addition of hydroxyethyl cellulose. The additional excipients were incorporated during the final blending stage to enhance flow properties, improve reconstitution behavior, and increase suspension stability of the coated granules.

The following examples (Examples 8 and 9) describe additional formulations that were prepared and evaluated during the development of the invention. These compositions were found to be operable within the scope of the present disclosure and demonstrate the feasibility of the claimed dosage forms. However, they were not determined to represent optimized or preferred formulations. The data are provided for illustrative purposes to show the range of ingredient combinations, concentrations, and preparation methods contemplated by the inventors. Accordingly, these examples should not be construed as limiting the invention to the specific embodiments described herein, but rather as evidence of the versatility and adaptability of the inventive concept across different formulation parameters.

### Example 8: Oral Solution

| | | Oral Solution | | | | | |
|---|---|---|---|---|---|---|---|
| | | C-1 | | C-2 | | C-3 | |
| S. No. | Ingredients | mg/mL | % | mg/mL | % | mg/mL | % |
| 1 | Amlodipine besylate | 2.000 | 0.200 | 2.000 | 0.200 | 2.000 | 0.200 |
| 2 | Benazepril hydrochloride | 8.000 | 0.800 | 8.000 | 0.800 | 8.000 | 0.800 |
| 3 | Butylated hydroxy anisole | 0.150 | 0.015 | 0.150 | 0.015 | 0.150 | 0.015 |
| 4 | Glycerin | 500.000 | 50.000 | 500.000 | 50.000 | 500.000 | 50.000 |
| 5 | Maltitol/Mannitol | 100.000 | 10.000 | 100.000 | 10.000 | 100.000 | 10.000 |
| 6 | Peppermint flavor | 0.500 | 0.050 | 0.500 | 0.050 | 0.500 | 0.050 |
| 7 | Purified water | 264.840 | 26.484 | 216.340 | 21.634 | 215.840 | 21.584 |
| 8 | Propylene glycol | 120.000 | 12.000 | 120.000 | 12.000 | 120.000 | 12.000 |
| 9 | Disodium EDTA | 0.500 | 0.050 | X | X | 0.500 | 0.050 |
| 10 | Hydroxyethyl cellulose | 1.000 | 0.100 | X | X | X | X |
| 11 | Hydroxy β-cyclodextrin/ | X | X | 50.000 | 5.000 | 50.000 | 5.000 |
| | povidone | | | | | | |
| 12 | Sodium benzoate | 1.000 | 0.100 | 1.000 | 0.100 | 1.000 | 0.100 |
| 13 | Citric acid monohydrate | 1.850 | 0.185 | 1.850 | 0.185 | 1.850 | 0.185 |
| 14 | Sodium citrate | 0.160 | 0.016 | 0.160 | 0.016 | 0.160 | 0.016 |

### Example 9: Powder/Pellet for Oral Solution

| S. No. | Ingredients | mg/mL | % |
|---|---|---|---|
| 1 | Amlodipine besylate | 2.000 | 1.352 |
| 2 | Benazepril hydrochloride | 8.000 | 5.407 |
| 3 | Butylated hydroxy anisole | 0.150 | 0.101 |
| 4 | Maltitol/Mannitol | 61.500 | 41.568 |
| 5 | Peppermint flavor | 0.500 | 0.338 |
| 6 | Disodium EDTA | 0.500 | 0.338 |
| 7 | Hydroxyethyl cellulose | 1.000 | 0.676 |
| 8 | Hydroxy β-cyclodextrin/povidone | 50.000 | 33.795 |
| 9 | Sodium benzoate | 1.000 | 0.676 |
| 10 | Citric acid monohydrate | 1.850 | 1.250 |
| 11 | Sodium citrate | 0.150 | 0.101 |
| 12 | Mannitol/sugar spheres | 8.500 | 5.745 |
| 13 | Hydroxypropyl methylcellulose | 8.500 | 5.745 |
| 14 | Isopropyl alcohol | Q.S. | Q.S. |
| 15 | Hydroxypropyl cellulose | 2.000 | 1.352 |
| 16 | Talc | 0.800 | 0.541 |
| 17 | Opadry clear (HPMC based) | 1.500 | 1.014 |

### Example 10: Powder/Pellet for Oral Suspension

| | | Powder for Oral Suspension | | | | | |
|---|---|---|---|---|---|---|---|
| | | C-1 | | C-2 | | C-3 | |
| S. No. | Ingredients | mg/mL | % | mg/mL | % | mg/mL | % |
| 1 | Amlodipine besylate | 2.000 | 1.189 | 2.000 | 1.144 | 2.000 | 1.144 |
| 2 | Benazepril hydrochloride | 8.000 | 4.754 | 8.000 | 4.577 | 8.000 | 4.576 |
| 3 | Butylated hydroxy anisole | 0.150 | 0.089 | 0.150 | 0.086 | 0.150 | 0.086 |
| 4 | Maltitol/Mannitol/Sucralose | 61.530 | 36.563 | 60.030 | 34.345 | 60.030 | 34.335 |
| 5 | Peppermint flavor | 0.500 | 0.297 | 0.500 | 0.286 | 0.500 | 0.286 |
| 6 | Disodium EDTA | 0.500 | 0.297 | 0.500 | 0.286 | 0.500 | 0.286 |
| 7 | Hydroxyethyl cellulose | 1.000 | 0.594 | 1.000 | 0.572 | 1.000 | 0.572 |
| | (optional) | | | | | | |
| 8 | Hydroxy β-cyclodextrin/ | 50.000 | 29.713 | 50.000 | 28.608 | 50.000 | 28.600 |
| | Povidone- (optional) | | | | | | |
| 9 | Sodium benzoate | 1.000 | 0.594 | 1.000 | 0.572 | 1.000 | 0.572 |
| 10 | Citric acid monohydrate | 1.850 | 1.099 | 1.850 | 1.058 | 1.850 | 1.058 |
| 11 | Sodium citrate | 0.150 | 0.089 | 0.150 | 0.086 | 0.150 | 0.086 |
| 12 | Mannitol/sugar spheres | 8.500 | 5.051 | 8.500 | 4.863 | 8.500 | 4.862 |
| | (optional) | | | | | | |
| 13 | Hydroxypropyl | 8.500 | 5.051 | 8.500 | 4.863 | 8.500 | 4.862 |
| | methylcellulose (optional) | | | | | | |
| 14 | Microcrystalline cellulose | 8.800 | 5.229 | 8.800 | 5.035 | 8.850 | 5.062 |
| 15 | Carboxymethylcellulose | 2.000 | 1.189 | 2.000 | 1.144 | 2.000 | 1.144 |
| | sodium | | | | | | |
| 16 | Crospovidone (optional)/ | 5.000 | 2.971 | 5.000 | 2.861 | 5.000 | 2.860 |
| | Colloidal silicon dioxide | | | | | | |
| 17 | Xanthan gum | 4.500 | 2.674 | 4.500 | 2.575 | 4.500 | 2.574 |
| 18 | Isopropyl alcohol | Q.S. | Q.S. | Q.S | Q.S. | Q.S. | Q.S. |
| 19 | Eudragit E 100 polymer | X | X | 8.000 | 4.577 | X | X |
| | (optional) | | | | | | |
| 20 | Resin (Kyron complex) | X | X | X | X | 8.000 | 4.576 |
| | (optional) | | | | | | |
| 21 | Hydroxypropyl cellulose | 2.000 | 1.189 | 2.000 | 1.144 | 2.000 | 1.144 |
| | (optional) | | | | | | |
| 22 | Talc (optional) | 0.800 | 0.475 | 0.800 | 0.458 | 0.800 | 0.458 |
| 23 | Opadry clear | 1.500 | 0.891 | 1.500 | 0.858 | 1.500 | 0.858 |
| | (HPMC based) (optional) | | | | | | |

### Example 11: Powder/Pellet for Oral Suspension

| S. No. | Ingredients | Function | Ranges % w/w | S1 | S2 |
|---|---|---|---|---|---|
| 1 | Benazepril hydrochloride | Active | 0.3-7 | 5.00 | 5.00 |
| | | pharmaceutical | | | |
| | | ingredient | | | |
| 2 | Amlodipine besylate | Active | 0.05-2 | 1.73 | 1.25 |
| | | pharmaceutical | | | |
| | | ingredient | | | |
| 3 | Maltitol/Mannitol/Sucralose | Sweetening agent | 10-75 | 20.66 | 14.34 |
| 4 | Hydroxy β-cyclodextrin | Complexing agent | 0.5-50 | 6.25 | 6.25 |
| 5 | Peppermint flavor | Flavouring agent | 0.05-3 | 0.63 | 0.63 |
| 6 | Butylated hydroxy anisole | Antioxidant | 0.01 to 2.20 | 0.32 | 0.31 |
| 7 | Sodium benzoate | Antimicrobial | 0.01 to 2.20 | 0.63 | 0.63 |
| | | preservative | | | |
| 8 | Citric acid monohydrate | Buffering agent | 0.05 to 10 | 6.25 | 5.00 |
| 9 | Sodium citrate | Buffering agent | 0.05 to 10 | 3.75 | 0.81 |
| 10 | Disodium EDTA | Chelating agent | 0.01-3 | 0.31 | 0.31 |
| 11 | Hydroxypropyl Methylcellulose | Coating agent | 1-15 | X | 7.53 |
| 12 | Microcrystalline cellulose | Suspending agent | 0.05-20 | 9.38 | 9.37 |
| 13 | Carboxymethylcellulose sodium | Suspending agent/ | 0.05-10 | 2.50 | 1.25 |
| | | Viscosity modifier | | | |
| 14 | Colloidal silicon dioxide/ | Anticaking agent | 0.05-10 | 3.13 | 3.13 |
| | Crospovidone | | | | |
| 15 | Xanthan gum | Gelling | 0.05-7 | 1.88 | 1.88 |
| | | agent/Suspending | | | |
| | | agent/Viscosity | | | |
| | | modifier | | | |
| 16 | Isopropyl alcohol/Purified water | Solvent/Vehicle | q.s | q.s | q.s |

### Example 12; Powder for Oral Suspension

| S. No. | Ingredients | Function | Ranges % w/w | S3 | S4 |
|---|---|---|---|---|---|
| 1 | Benazepril hydrochloride | Active | 0.3-7 | 5.00 | 5.00 |
| | | pharmaceutical | | | |
| | | ingredient | | | |
| 2 | Amlodipine besylate | Active | 0.05-2 | 1.73 | 1.25 |
| | | pharmaceutical | | | |
| | | ingredient | | | |
| 3 | Maltitol/Mannitol/Sucralose | Sweetening agent | 10-75 | 20.66 | 14.34 |
| 4 | Hydroxy β-cyclodextrin | Complexing | 0.5-50 | 6.25 | 6.25 |
| | | agent | | | |
| 5 | Peppermint flavor | Flavouring agent | 0.05-3 | 0.63 | 0.63 |
| 6 | Butylated hydroxy anisole | Antioxidant | 0.01 to 2.20 | 0.32 | 0.31 |
| 7 | Sodium benzoate | Antimicrobial | 0.01 to 2.20 | 0.63 | 0.63 |
| | | preservative | | | |
| 8 | Citric acid monohydrate | Buffering agent | 0.05 to 10 | 6.25 | 5.00 |
| 9 | Sodium citrate | Buffering agent | 0.05 to 10 | 3.75 | 0.81 |
| 10 | Disodium EDTA | Chelating agent | 0.01-3 | 0.31 | 0.31 |
| 11 | Hydroxypropyl Methylcellulose | Coating agent | 1-15 | X | 7.53 |
| 12 | Microcrystalline cellulose | Suspending agent | 0.05-20 | 9.38 | 9.37 |
| 13 | Carboxymethylcellulose sodium | Suspending | 0.05-10 | 2.50 | 1.25 |
| | | agent/ | | | |
| | | Viscosity | | | |
| | | modifier | | | |
| 14 | Colloidal silicon dioxide/ | Anticaking agent | 0.05-10 | 3.13 | 3.12 |
| | Crospovidone | | | | |
| 15 | Xanthan gum | Gelling | 0.05-7 | 1.88 | 1.88 |
| | | agent/Suspending | | | |
| | | agent/Viscosity | | | |
| | | modifier | | | |
| 16 | Isopropyl alcohol/Purified water | Solvent/Vehicle | q.s | q.s | q.s |
| 17 | Dibasic calcium phosphate | Diluent | X | 37.36 | X |
| | anhydrous | | | | |
| 18 | Dimethicone | Antifoaming | X | 0.13 | 0.13 |
| | | agent | | | |

### General Manufacturing Process of Powder for Oral Suspension

### Preparation of Amlodipine Part

Example 12 was conducted in accordance with the general procedure described in Example 2 for the preparation of the amlodipine part, except for the following modifications. In step 1, dibasic calcium phosphate anhydrous, sodium citrate, and sodium benzoate were sifted in place of maltitol, mannitol, and/or sucralose. In step 3, amlodipine besylate and dimethicone were added to the dispersion obtained in step 2 and mixed until a uniform, lump-free dispersion was achieved. The remaining process parameters and steps were performed substantially as described in Example 2 to yield the modified amlodipine granules.

### Preparation of Benazepril Hydrochloride Part by Wet Granulation Technique/Blending:

The benazepril hydrochloride part was prepared by a wet granulation process generally in accordance with standard granulation procedures, as described below.
1. Sifting and Blending of Excipients: Maltitol, mannitol, and/or sucralose, microcrystalline cellulose, sodium carboxymethyl cellulose, disodium edetate, butylated hydroxyanisole, and xanthan gum were sifted through an appropriate mesh and transferred into a rapid mixer granulator (RMG).
2. Preparation of Binder Solution: The required quantity of an isopropyl alcohol and purified water mixture was dispensed into a stainless-steel vessel. Dimethicone was added under stirring and mixed until a clear, lump-free solution was obtained.
3. Dry Mixing: The sifted materials from step 1 were mixed in the RMG for approximately 10 minutes with the impeller on and chopper off.
4. Granulation: The binder solution from step 2 was slowly added to the dry blend obtained in step 3 with the impeller on and chopper off.
5. Kneading: The wet mass obtained in step 4 was kneaded using both the impeller and chopper to form uniform wet granules.
6. Adjustment of Granulation Solvent (if required): If necessary, additional isopropyl alcohol and purified water mixture was added to achieve proper granulation consistency, followed by further kneading with the impeller and chopper on.
7. Drying: The wet mass from step 5 was transferred to a fluid bed dryer (FBD) bowl and dried at a controlled temperature until the desired loss on drying (LOD) not more than 2.0% w/w was achieved.
8. Sizing of Dried Granules: The dried granules were passed through a sifting machine to obtain uniform granule size distribution and collected.
9. Pre-Blending: The sifted granules obtained in step 8 were transferred into a blender for pre-blending.
10. Final Blending with Drug and Flavoring Agents: Benazepril hydrochloride, citric acid monohydrate, colloidal silicon dioxide, and peppermint flavor were sifted separately and added to the blender containing the granules from step 9. The mixture was blended for approximately 10 minutes to ensure uniform distribution.
11. Final Blending and Filling: The amlodipine granules (prepared as described in Example 12) were transferred to the blender containing the benazepril granules from step 10 and blended for an additional 10 minutes to obtain a homogeneous mixture. The final blend was filled into amber polyethylene terephthalate (PET) bottles under controlled environmental conditions.

### Preparation of Amlodipine Part by Top Spray Technique:

The Amlodipine part was prepared by a top spray coating process using a fluid bed processor as follows.
1. Pre-warming of Substrate: Microcrystalline cellulose was sifted through an appropriate mesh and transferred into a fluid bed processor. The material was pre-warmed to a product temperature of 30±5° C. for approximately 10 minutes.
2. Preparation of Coating Solution I: A weighed quantity of an isopropyl alcohol and purified water mixture was transferred into a stainless-steel vessel. Under continuous stirring, butylated hydroxyanisole, hydroxy β-cyclodextrin, amlodipine besylate, and dimethicone were sequentially dispersed and mixed until a uniform and homogeneous solution was obtained.
3. Primary Coating: The pre-warmed material from step 1 was coated with the solution prepared in step 2 using a top spray technique until the desired weight gain was achieved. The coated granules were then dried within the same equipment under controlled conditions.
4. Sifting: The coated granules obtained from step 3 were sifted through an appropriate mesh to achieve uniform particle size and collected separately.
5. Pre-warming for Secondary Coating: The granules from step 4 were transferred back into the fluid bed processor and pre-warmed to a product temperature of 30±5° C. for approximately 10 minutes.
6. Preparation of Coating Solution II: A fresh isopropyl alcohol and purified water mixture was dispensed into a stainless-steel vessel. Hydroxypropyl methylcellulose was slowly dispersed into the mixture under stirring and mixed until a uniform solution was obtained.
7. Secondary Coating: The pre-warmed granules from step 5 were coated with the hydroxypropyl methylcellulose solution prepared in step 6 using the top spray technique until the desired weight gain was achieved.
8. Final Sifting: The coated granules obtained from step 7 were sifted to remove any agglomerates and collected separately. The resulting granules were uniform, free-flowing, and suitable for subsequent blending and filling operations.

### Benazepril Hydrochloride Part—Processing by Wet Granulation Technique-Blending:

The Benazepril hydrochloride part was prepared by a wet granulation process followed by blending as described below.
1. Sifting and Loading of Excipients: Maltitol, mannitol, and/or sucralose, microcrystalline cellulose, sodium carboxymethyl cellulose, disodium edetate, butylated hydroxyanisole, and xanthan gum were sifted through an appropriate mesh and transferred into a rapid mixer granulator (RMG).
2. Preparation of Binder Solution: The required quantity of an isopropyl alcohol and purified water mixture was dispensed into a stainless-steel vessel. Dimethicone was added under continuous stirring until a clear, lump-free solution was obtained.
3. Dry Mixing: The sifted materials from step 1 were mixed in the RMG for approximately 10 minutes with the impeller on and chopper off to ensure uniform distribution.
4. Granulation: The binder solution prepared in step 2 was gradually added to the dry blend from step 3 with the impeller on and chopper off. Mixing continued until the mass achieved suitable wet granulation consistency.
5. Kneading: The wet mass obtained in step 4 was kneaded using both the impeller and chopper until uniform granules were formed.
6. Adjustment of Granulation Solvent (if required): If necessary, additional isopropyl alcohol and purified water mixture was added to maintain the desired granulation consistency, followed by further kneading with the impeller and chopper on.
7. Drying: The wet mass obtained was transferred to a fluid bed dryer (FBD) bowl and dried at a controlled temperature until a loss on drying (LOD) not exceeding 2.0% w/w was achieved.
8. Sizing and Collection of Dried Granules: The dried granules were passed through a sifting machine to ensure uniform particle size and then collected.
9. Pre-Blending: The sifted granules obtained in step 8 were transferred into a blender for pre-blending to achieve homogeneity.
10. Final Blending with Drug and Flavoring Agents: Benazepril hydrochloride, citric acid monohydrate, colloidal silicon dioxide, and peppermint flavor were sifted through an appropriate mesh, added to the pre-blended granules from step 9, and blended for approximately 10 minutes to obtain a uniform mixture.
11. Final Combination and Filling: The Amlodipine granules (prepared as described in Example [X]) were transferred into the blender containing the benazepril granules from step 10 and blended for an additional 10 minutes to ensure homogeneity. The final blended granules were filled into amber polyethylene terephthalate (PET) bottles under controlled environmental conditions to yield the finished product.

The resulting granules exhibited uniform flow characteristics, consistent blend uniformity, and satisfactory stability, rendering them suitable for use in oral reconstitution formulations.

1. A composition for a stable oral liquid formulation comprising:
benazepril or a pharmaceutically acceptable salt or solvate thereof;
amlodipine or a pharmaceutically acceptable salt or solvate thereof;
a sweetening agent; and
a complexing agent.

2. The composition according to claim 1,
wherein the benazepril or a pharmaceutically acceptable salt or solvate thereof is in an amount of from about 0.3% to about 7% w/w;
wherein the amlodipine or a pharmaceutically acceptable salt or solvate thereof is in amount of from about 0.05% to about 2% w/w;
wherein the sweetening agent is in an amount of from about 0.5% to about 75% w/w; and
wherein the complexing agent is in an amount of from about 0.5% to about 50% w/w.

3. The composition according to claim 1, wherein the sweetening agent is selected from the group consisting of glucose, fructose, sucrose, xylitol, tagatose, sucralose, maltitol, allulose, isomaltulose, isomalt, lactitol, sorbitol, erythritol, liquid glucose, compressible sugar, confectioner's sugar, dextrose, maltitol solution, a maltitol oligomer, maltose, mannitol, neohesperidin dihydrochalcone, thaumatin, trehalose, magnasweet 110, magnasweet 180, xylitol, sorbitan monoleate, trehalose, maltodextrin, polydextrose, glycerin, inulin, maltol, acesulfame and and its salts, alitame, aspartame, neotame, sodium cyclamate, saccharin and its salts, neohesperidin dihydrochalcone, stevioside, thaumatin, hydrogenated starch hydrolysates, maltitol syrup, high fructose corn syrup, sweet AM liquid/powder, prosweet, maltisweet, Sorbo®, invertose, rebalance M60 and X60, ora-sweet, ora-sweet-SF, monkfruit, flavored syrups, and the like, and combinations thereof.

4. The composition according to claim 1, wherein the complexing agent is selected from the group consisting of ethylenediaminetetraacetic acid, cyclodextrins, substituted cyclodextrins, alkali metal salts of partly substituted carboxyalkylcyclodextrins or partly substituted water-soluble alkylcyclodextrins, hydroxypropyl-β-cyclodextrins, sulfobutylether-β-cyclodextrins, methylated β-cyclodextrins, RAMEB (statistically methylated or randomly-methylated-β-cyclodextrin β-cyclodextrin), TRIMEB (heptakis (2,3,6-tri-O-methyl)-β-cyclodextrin), CRYSMEB (weakly methylated β-cyclodextrin), phosphates, zeolites, ammonia, EDTA and their salts, tartaric acid, citric acid, polyvinylpyrrolidone, polyethylene glycol and its derivatives, cross linked polymers such as carbomers, crosslinked polyvinylpyrrolidone, crosslinked sodium carboxyl methyl cellulose, polyacrylamide, cross linked hydroxyl propyl methyl cellulose, polyethylene glycol diacrylate, cross linked chitosan, and the like, and combinations thereof.

5. The composition according to claim 1, wherein the viscosity modifier is selected from the group consisting of hydrocolloid gums, cellulose derivatives, hydroxyethylcellulose, methylcellulose, carboxymethylcellulose, carbomers, polyvinyl alcohols, silicon dioxide, and the like, and combinations thereof.

6. The composition according to claim 1, further comprising at least one of a suspension aid, suspension stabilizer, gelling agent, thickener selected from the group consisting of dextrin, cellulose derivatives, starches, pectin, polyethylene glycol, polyethylene oxide, trehalose, certain silicates, gums, silicon dioxide, microcrystalline cellulose, polyvinylpyrrolidone, 3-butoxy-2-hydroxypropylhydroxyethylcellulose, acrylamide homo- and copolymers, acrylic acid homo- and copolymer, alginates, carboxymethylhydroxyethylcellulose, carboxy-vinyl copolymers, cellulose, microcrystalline cellulose, hydrophobically modified hydroxyethylcellulose, hydroxypropyl guar, hydroxypropyl methylcellulose, hydroxypropylcellulose, partially and fully hydrolyzed polyvinyl alcohols, partially neutralized polyacrylic acid, polyalkylene glycol, polysaccharide gums, polyvinylpyrrolidones, starches, vinylpyrrolidone homo- and copolymers, water-soluble cellulose ethers, bentonite, laponites, kaolinite, dickite, nacrite, pyrophylite, talc, vermiculite, sauconite, saponite, nontronite, montmorillonite, organically modified montmorillonite clays, and the like, and combinations thereof in an amount of from about 0.05 to 30% w/w.

7. The composition according to claim 1, further comprising one or more pharmaceutically acceptable excipients selected from at least one of a flavoring agent, a preservative/antioxidant, a buffering agent, a suspending agent, a solvent/vehicle, a cosolvent, a chelating agent, and combinations thereof.

8. The composition according to claim 1, wherein the stable oral liquid formulation has a pH of about 3 to about 7.5.

9. A method of treating a patient in need of therapy for hypertension or related cardiovascular disease, comprising administering a therapeutically effective amount of the oral liquid formulation prepared according to claim 1.

10. A composition for a stable oral liquid formulation comprising:
benazepril or a pharmaceutically acceptable salt or solvate thereof;
amlodipine or a pharmaceutically acceptable salt or solvate thereof;
a sweetening agent;
a complexing agent;
a viscosity modifier; and
an anticaking agent.

11. The composition according to claim 10, further comprising a suspending agent selected from methylcellulose, microcrystalline cellulose, sodium carboxymethylcellulose, acacia, xanthan gum, gellan gum, bentonite, and the like, or their combinations of two or more.

12. The composition according to claim 10,
wherein the benazepril or a pharmaceutically acceptable salt or solvate thereof is in an amount of from about 0.3% to about 7% w/w;
wherein the amlodipine or a pharmaceutically acceptable salt or solvate thereof is in an amount of from about 0.05% to about 2% w/w;
wherein the sweetening agent is in an amount of from about 0.5% to about 75% w/w;
wherein the complexing agent is in an amount of from about 0.5% to about 50% w/w;
wherein the viscosity modifier is in an amount of from about 0.05% to about 10% w/w;
wherein the anticaking agent is in an amount of from about 0.05% to about 10% w/w; and
wherein the suspending agent is in an amount of from 0.5% to about 20% w/w.

13. The composition according to claim 10,
wherein the sweetening agent is selected from the group consisting of maltitol, mannitol, sucralose and combinations thereof;
wherein the complexing agent is a cyclodextrin; and
wherein the viscosity modifier is carboxymethyl cellulose.

14. The composition according to claim 10, wherein the amlodipine or a pharmaceutically acceptable salt or solvate thereof is in the form of uniform granules prepared by adding amlodipine and optionally dimethicone to a binder solution including the complexing agent and a stabilizer to obtain a dispersion, granulating and drying the dispersion and milling and sieving to obtain the uniform granules.

15. The composition according to claim 14, wherein the benazepril or a pharmaceutically acceptable salt or solvate thereof is in the form of uniform coated granules prepared by dissolving benazepril in the solvent and coating onto inert spheres by a bottom spray or top spray process, incorporating at least one excipient to the solvent, and drying and blending to obtain the uniform coated granules.

16. A method of preparing a stable oral liquid pharmaceutical formulation comprising:
dissolving or dispersing a complexing agent in a pharmaceutically acceptable solvent to obtain a first clear solution or a first uniform dispersion;
adding a calcium channel blocker or a pharmaceutically acceptable salt thereof to the first clear solution or the first uniform dispersion and mixing until dissolution or dispersion is achieved;
adding a preservative and/or antioxidant to the first clear solution or the first uniform dispersion obtained and mixing until uniform;
dissolving or dispersing an angiotensin converting enzyme inhibitor or a pharmaceutically acceptable salt thereof in a solvent to obtain a second clear solution or a second uniform dispersion;
separately dissolving or dispersing a viscosity modifier in a co-solvent to obtain a thickening solution or dispersion;
combining the first and second clear solutions or the first and second uniform dispersion with the thickening solution or dispersion under continuous stirring to obtain a homogenous mixture;
incorporating a sweetening agent to the homogenous mixture; and
adjusting the final volume with a solvent to obtain a stable oral liquid formulation.

17. The method of claim 16, wherein the angiotensin converting enzyme inhibitor is benazepril or a pharmaceutically acceptable salt or solvate thereof, and the calcium channel blocker is amlodipine or a pharmaceutically acceptable salt or solvate thereof.

18. The method of claim 16, wherein the complexing agent is a cyclodextrin, the viscosity modifier is hydroxyethyl cellulose, and the sweetening agent is maltitol, mannitol, or sucralose.

19. The method of claim 16, wherein the formulation has a pH of about 3 to about 7.5.

20. The method of claim 16, wherein:
the angiotensin converting enzyme inhibitor is in an amount of from about 0.3% to about 7% w/w;
the channel blocker or a pharmaceutically acceptable salt or solvate thereof is in amount of from about 0.05% to about 2% w/w;
the sweetening agent is in an amount of from about 0.5% to about 75% w/w; and
the complexing agent is in an amount of from about 0.5% to about 50% w/w.
